(19) United States
(12) Patent Application Publication
(10) Pub. No.: US 2026/0256576 A1
(43) Pub. Date: Sep. 3, 2026

(54) DOCKING STATION FOR CIRCULATORY ASSIST DEVICES

(71) Applicant: CardiacBooster B.V., Nijmegen (NL)

(72) Inventors: Florian Niklas Ludwig, Hilversum (NL); Daniël Immanuel Michaël van Dort, Nijmegen (NL); Patrick Griffin, Galway (IE)

(21) Appl. No.: 19/546,062

(22) Filed: Feb. 20, 2026

Related U.S. Application Data
(63) Continuation of application No. PCT/IB2024/058254, filed on Aug. 24, 2024.
(60) Provisional application No. 63/578,516, filed on Aug. 24, 2023.

(51) Int. Cl. A61F 2/24

(52) U.S. Cl. CPC A61F 2/2418; A61F 2/2433; A61F 2210/0004; A61F 2210/0014

(57) ABSTRACT

In some variations, a medical device may include an expandable support with an interior region and a seal arranged within the interior region of the expandable support. The expandable support may have a delivery configuration with a first diameter and a deployment configuration with a second diameter larger than the first diameter. The seal may include a seal opening and a sealing element configured to seal around a circulatory assist device seated in the seal opening. In some variations, the medical device may be delivered to a treatment site in a cardiovascular lumen (e.g., descending aorta) of a patient, and the circulatory assist device may be operated while positioned in the seal opening to provide circulatory support to the patient.

### CROSS-REFERENCE TO RELATED APPLICATIONS

The present application is a continuation of International Application No. PCT/IB2024/058254, filed Aug. 24, 2024, which claims the benefit of priority to U.S. Provisional Patent Application No. 63/578,516, filed Aug. 24, 2023, which is incorporated by reference herein in its entirety.

### TECHNICAL FIELD

The present technology relates to a docking station for circulatory assist devices.

### BACKGROUND

For some patients, such as those suffering from cardiogenic shock or decompensated heart failure, or those undergoing high-risk percutaneous coronary interventions (PCI), a patient's heart function may be compromised such that the use of circulatory assist devices may be required to maintain adequate blood flows through the circulatory system.

One type of circulatory assist device is transvalvular percutaneous mechanical cardiac support devices (pMCS), which are configured to be placed across the aortic valve. The placement of such transvalvular devices, however, carries the risk of endovascular or embolic injury, and so typically must be performed by a highly trained and skilled interventional cardiologist who can successfully navigate the devices over the aortic arch and across the aortic valve without incurring tissue damage. However, this level of skill is not always present in interventional cardiologists who may be inexperienced. Additionally, the clinical need for circulatory assist devices may arise in situations in which skilled operators are not available (e.g., emergency situations, such as in an ambulance or peripheral acute chest pain intervention units).

What is needed, therefore, are improved circulatory support systems and methods.

### SUMMARY

The present technology is illustrated, for example, according to various aspects described below, including with reference to FIGS. 1A-13B. Various examples of aspects of the present technology are described as numbered clauses (1, 2, 3, etc.) for convenience. These are provided as examples and do not limit the present technology.

Example A1. A medical device, comprising:
an expandable support having a delivery configuration with a first diameter and a deployed configuration with a second diameter larger than the first diameter, wherein the expandable support comprises an interior region; and
a seal arranged within the interior region of the expandable support, wherein the seal comprises a seal opening and a sealing element configured to seal around a circulatory assist device seated in the seal opening.

Example A2. The medical device of Example A1, wherein the seal opening has a variable diameter.

Example A3. The medical device of Example A1, wherein the seal opening has a fixed diameter.

Example A4. The medical device of any one of Examples A1-A3, wherein the seal opening is radially centered within the interior region of the expandable support.

Example A5. The medical device of any one of Examples A1-A3, wherein the seal opening is radially offset within the interior region of the expandable support.

Example A6. The medical device of any one of Examples A1-A5, wherein the sealing element comprises a plurality of leaflets.

Example A7. The medical device of any one of Examples A1-A6, wherein the seal comprises a valve.

Example A8. The medical device of Example A7, wherein the valve is a tricuspid valve.

Example A9. The medical device of any one of Examples A1-A8, wherein the expandable support is self-expandable.

Example A10. The medical device of Example A9, wherein the expandable support comprises a stent.

Example A11. The medical device of Example A10, wherein the stent has an outwardly flared shape.

Example A12. The medical device of Example A10, wherein the stent has a toroidal shape.

Example A13. The medical device of any one of Examples A1-A12, wherein the expandable support comprises a shape memory material.

Example A14. The medical device of any one of Examples A1-A13, wherein the expandable support comprises an inflatable member.

Example A15. The medical device of any one of Examples A1-A14, further comprising a first pressure sensor configured to measure a pressure on an upstream side of the seal, and a second pressure sensor configured to measure a pressure on a downstream side of the seal.

Example A16. The medical device of any one of Examples A1-A15, further comprising an outer sheath comprising a lumen configured to hold the expandable support in the delivery configuration.

Example A17. The medical device of any one of Examples A1-A16, further comprising an elongate member coupled to the expandable support configured for one or more of delivery or retrieval of the medical device.

Example A18. The medical device of Example A17, wherein the elongate member is removably coupled to the expandable support.

Example A19. The medical device of any one of Examples A1-A18, wherein the expandable support comprises an engagement feature configured to facilitate retrieval of the medical device from a treatment site.

Example A20. The medical device of Example A19, wherein the engagement feature comprises at least one of a knob, loop, hook, or mating feature, coupled to a downstream surface of the expandable support.

Example A21. The medical device of any one of Examples A1-A20, wherein the expandable support comprises a bioabsorbable material.

Example A22. The medical device of any one of Examples A1-A21, wherein the expandable support is configured to transition to a flow restoration configuration in which a diameter of the interior region is larger than the seal opening.

Example A23. A system, comprising:
the medical device of any one of Examples A1-A22; and
a circulatory assist device configured to be seated within the seal opening.

Example A24. The system of Example A23, wherein the circulatory assist device comprises a conduit and a volume displacement member arranged within the conduit.

Example A25. The system of Example A24, wherein the volume displacement member comprises a balloon configured to be cyclically inflated and deflated.

Example A26. The system of Example A23, wherein the circulatory assist device comprises an impeller device.

Example A27. The system of Example A1-A26, wherein the interior region comprises a first channel and a second channel, wherein the sealing element is disposed in the first channel, the second channel being configured to receive an intravascular device.

Example A27. The system of Example A27, wherein the second channel comprises a second sealing element configured to fluidly seal around the intravascular device.

Example B1. A method of providing circulatory assistance, comprising:
delivering a medical device to a treatment site in a vascular lumen of a patient, wherein the medical device comprises an expandable support and a seal arranged within an interior region of the expandable support, wherein the seal comprises a seal opening;
positioning a circulatory assist device in the seal opening;
forming a seal between the circulatory assist device and the expandable support with the seal; and
operating the circulatory assist device while the circulatory assist device is positioned in the seal opening.

Example B2. The method of Example B1, wherein the seal opening has a variable diameter.

Example B3. The method of Example B1, wherein the seal opening has a fixed diameter.

Example B4. The method of any one of Examples B1-B3, further comprising forming an outer seal between the medical device and an inner wall of the vascular lumen.

Example B5. The method of Example B4, wherein forming the outer seal comprises allowing the expandable support to self-expand against the inner wall of the vascular lumen.

Example B6. The method of any one of Examples B1-B5, wherein forming the outer seal comprises actively expanding the expandable support against the inner wall of the vascular lumen.

Example B7. The method of any one of Examples B1-B6, further comprising repositioning the circulatory assist device at least partially in a left ventricle of the patient to increase circulatory support for the patient.

Example B8. The method of Example B7, further comprising repositioning the circulatory assist device in the vascular lumen of the patient to decrease circulatory support for the patient.

Example B9. The method of Example B8, wherein the seal comprises a valve.

Example B10. The method of Example B9, further comprising measuring a pressure gradient between an upstream side of the valve and a downstream side of the valve.

Example B11. The method of Example B10, further comprising controlling the circulatory assist device based at least in part on the measured pressure gradient.

Example B12. The method of any one of Examples B1-B11, wherein the medical device further comprises an elongate member coupled to the expandable support, wherein the method further comprises decoupling the elongate member from the expandable support after delivering the medical device to the treatment site.

Example B13. The method of any one of Examples B1-B12, wherein positioning the circulatory assist device in the seal opening is performed after delivering the medical device to the treatment site in the vascular lumen.

Example B14. The method of any one of Examples B1-B12, wherein positioning the circulatory assist device in the seal opening is performed before delivering the medical device to the treatment site in the vascular lumen.

Example B15. The method of any one of Examples B1-B14, wherein delivering the medical device to the treatment site is performed without imaging guidance.

Example B16. The method of any one of Examples B1-B15, comprising allowing the medical device to remain at the treatment site for a treatment period of at least one week.

Example B17. The method of Example B16, comprising allowing the medical device to remain at the treatment site for a treatment period of at least two weeks.

Example B18. The method of Example B17, comprising allowing the medical device to remain at the treatment site for a treatment period of at least one month.

Example B19. The method of any one of Examples B1-B18, wherein the circulatory assist device is a percutaneous ventricular assist device (pVAD).

Example B20. The method of any one of Examples B1-B19, wherein the vascular lumen is a descending aorta.

Example B21. The method of any one of Examples B1-B19, wherein the vascular lumen is a pulmonary artery.

Example C1. A medical device, comprising:
an expandable support having a delivery configuration with a first outer diameter and a deployed configuration with a second outer diameter larger than the first outer diameter, wherein the expandable support comprises a lumen extending therethrough,
wherein the expandable support is configured to seal with a circulatory assist device seated in the lumen.

Example C2. The medical device of Example C1, wherein the lumen is configured to interact with a sealing element on the circulatory assist device.

Example C3. The medical device of Example C1 or C2, wherein the expandable support has a first lumen diameter in the delivery configuration and a second lumen diameter in the deployed configuration, wherein the second lumen diameter is smaller than the first lumen diameter.

Example C4. The medical device of Example C3, wherein an interior region of the expandable support comprises an inflatable member.

Example C5. The medical device of any one of Examples C1-C4, further comprising a sealing member configured to couple to the expandable support.

Example C6. The medical device of Example C5, wherein the sealing member comprises an inflatable member.

Example D1. A method of providing circulatory assistance, comprising:
delivering a medical device to a treatment site in a vascular lumen of a patient, wherein the medical device comprises a medical device of any one of Examples C1-C6;
positioning a circulatory assist device in the lumen;
forming a seal between the circulatory assist device and the expandable support; and
operating the circulatory assist device while the circulatory assist device is positioned in the lumen.

Example D2. The method of Example D1, wherein forming a seal comprises engaging a sealing element on the circulatory assist device with the lumen.

Example D3. The method of Example D1 or D2, wherein forming a seal comprises delivering a sealing member separate from the expandable support to the treatment site.

Example D4. The method of any one of Examples D1-D3, wherein the circulatory assist device comprises a conduit and a volume displacement member arranged within the conduit.

Example D5. The method of Example D4, wherein the volume displacement member comprises a balloon configured to be cyclically inflated and deflated.

Example D6. The method of any one of Examples D1-D3, wherein the circulatory assist device comprises an impeller device.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIG. 1A is an illustrative schematic of an example circulatory assist device, in accordance with the present technology.

FIGS. 1B-1F are illustrative schematics of an example circulatory assist device during operation, in accordance with the present technology.

FIGS. 2A and 2B are illustrative schematics of an aortic pump docking station in a delivery configuration and a deployed configuration, respectively, in accordance with the present technology.

FIG. 2C is an illustrative schematic of an aortic pump docking station in accordance with the present technology.

FIG. 3 is an illustrative schematic of an example aortic pump docking station, in accordance with the present technology.

FIG. 4 is an illustrative schematic of an example aortic pump docking station, in accordance with the present technology.

FIGS. 5A-5C are illustrative schematics of various example aortic pump docking stations, in accordance with the present technology.

FIG. 5D is an illustrative schematic of an example aortic pump docking station positioned in a descending aorta, in accordance with the present technology.

FIGS. 6A-6D are illustrative schematics of various example aortic pump docking stations, in accordance with the present technology.

FIG. 7 is a schematic flowchart of an example method of operating an aortic pump docking station, in accordance with the present technology.

FIGS. 8A-8H are illustrative schematics of various aspects of an example method of operating an aortic pump docking station, in accordance with the present technology.

FIGS. 9A-9D are illustrative schematics of various aspects of an example method of operating an aortic pump docking station, in accordance with the present technology.

FIGS. 10A-10E are illustrative schematics of a method of operating an example aortic pump docking station, in accordance with the present technology.

FIGS. 11A-11D are illustrative schematics of a method of operating an example aortic pump docking station, in accordance with the present technology.

FIGS. 12A-12E are illustrative schematics of a method of operating an example artic pump docking station, in accordance with the present technology.

FIGS. 13A and 13B are illustrative schematics of an example docking station configured to receive multiple devices.

### DETAILED DESCRIPTION

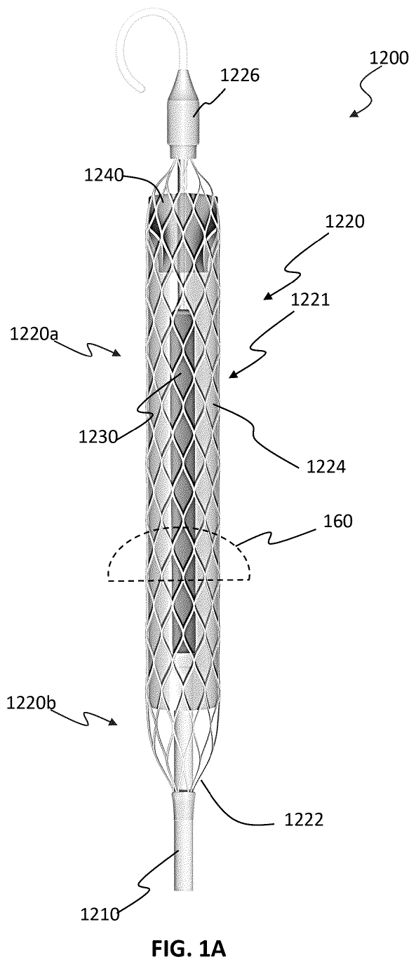

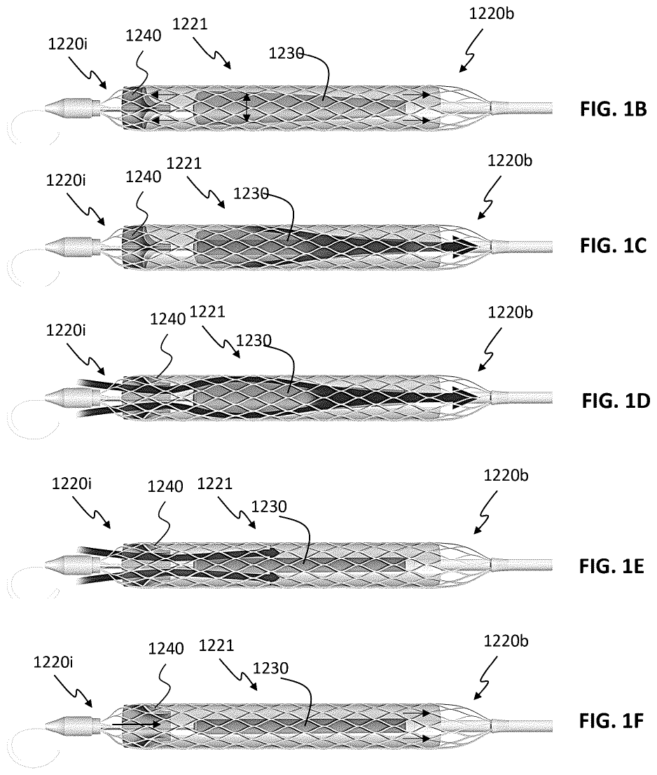

The present technology relates to circulatory assist systems and methods. Some aspects of the present technology, for example, are directed to a docking station for cardiac assist devices and methods. A docking station can, for example, be delivered percutaneously into a cardiovascular lumen and be operably combined with a circulatory assist device capable of pumping blood at flows high enough to support patients in cardiogenic shock, acute myocardial infarction, acute heart failure or during high-risk percutaneous coronary interventions, or other situations requiring hemodynamic support with reduced levels of hemolysis. Specific details of several aspects of the technology are described below with reference to FIGS. 1A-13B.

As used herein, the terms “proximal” and “distal” (and derivatives thereof) are used primarily within a frame of reference of a user placing a circulatory assist device within a patient, unless otherwise specified. For example, “proximal” primarily refers to a direction closer to the user, while “distal” primarily refers to a direction farther from the user. The term “upstream” in the vascular system means vascular locations closer to the patient's heart, while “downstream” means vascular locations further away from the patient's heart, regardless of the direction of blood flow through the vessel at the relevant time.

The circulatory assist devices and systems of the present technology may be used to provide circulatory assistance (e.g., cardiac assistance) in a variety of procedures and to address a variety of patient conditions. For example, the circulatory assist devices and systems may be used for cardiac assist during high-risk percutaneous coronary interventions (PCI) including angioplasty and stenting. Furthermore, the circulatory assist devices and systems may be used to provide cardiac support for patients experiencing cardiogenic shock or heart failure (e.g., decompensated heart failure). Furthermore, the circulatory assist devices and systems may be used to provide cardiac support for patients experiencing acute myocardial infarction, during surgery or other medical procedures, etc. For example, for such procedures, the circulatory assist devices may be configured for placement at least partially in the descending aorta. In some procedures, the circulatory assist devices can be additionally or alternatively configured for placement at least partially in the left ventricle. However, placement at various other cardiovascular lumen sites is also possible, including at least partially in the ascending aorta, the right atrium, right ventricle, and/or pulmonary artery.

A variety of circulatory assist devices, and/or other medical devices such as other intravascular devices may be used in conjunction with the docking station in accordance with the present technology. FIG. 1A illustrates an example circulatory assist device 1200 (e.g., an example of circulatory assist device 10). The circulatory assist device 1200 may include a pump arrangement 1220 having a pump body 1221 with a pump region 1220a configured to receive blood, an outflow region 1220b, and a pump 1230 in the pump region and configured to drive the received blood toward the outflow region 1220b. In some variations, the circulatory assist device 1200 may further include a sealing element 1260 configured to form a peripheral seal between the circulatory assist device 1200 (e.g., the pump body 1221) against a seal opening or other opening of the docking station 100. However, in some variations the sealing element 1260 may be omitted from the circulatory assist device 1200.

As further described herein, the pump body 1221 may further include at least one inlet valve 1240 configured to receive a fluid through the inlet of the conduit along the flow axis, and a pump 1230 arranged in the conduit. The inlet valve 1240 may, for example, include a multi-leaflet valve, such as a tri-leaflet valve. Examples of suitable inlet valves for the pump body 1221 are described in further detail in U.S. Provisional Patent Application No. 63/591,900, which is incorporated in its entirety herein by reference. The pump 1230 may, for example, include a volume displacement member, impeller, or other suitable pump mechanism. The volume displacement member, if present, may be operable in an expansion phase and a contraction phase. For example, in some variations the volume displacement member may include a balloon, and the balloon may be inflated in the expansion phase, and deflated in the contraction phase. However, the pump may omit an outlet valve that would be configured to convey fluid from the outflow region 1220b, away from the pump arrangement 1220, and to the cardiovascular lumen.

The circulatory assist device 1200 is characterized by axial flow between the inlet and the outlet of the conduit. In other words, in some variations, fluid pumped by the circulatory assist device 1200 travels from the inlet to the outlet substantially entirely or predominantly axially along (e.g., aligned with) the flow axis of the conduit. In some variations, the fluid flow in the conduit has limited to no radial flow component., and/or limited to no circumferential flow component. The flow axis of the conduit may be substantially coincident with a longitudinal axis of the conduit, for example, though it should be understood that axial flow includes both flow of fluid coincident with the longitudinal axis and flow of fluid generally parallel to the longitudinal axis. The circulatory assist device 1200 with axial flow may have a number of advantages. For example, because forces acting on the fluid within the pump body are generally oriented in the same direction, the fluid travels in a linear path through the circulatory assist device 1200 and experiences less turbulence, thereby resulting in less disturbance in components of the fluid itself (e.g., less hemolysis in blood pumped by the circulatory assist device 1200). Additionally, since flow occurs all in the same general axial direction (e.g., with little to no radial flow component), the kinetic behavior of the pump body (e.g., expansion and contraction of the volume displacement member, such as inflation and deflation of a balloon) can be more streamlined and energy efficient.

FIGS. 1B-1F illustrate various phases of operation in which fluid may be allowed to exit the conduit of the pump body via maintained momentum during the expansion phase and at least a part of the contraction phase of a pump 1230. Although the pump 1230 is primarily shown and described below as a balloon, it should be understood that the same principles of operation apply with respect to other variations of circulatory assist devices that include different kinds of pumps 1230 (e.g., impeller).

FIG. 1B illustrates a pump body 1221 that has received fluid (e.g., blood) through the inlet valve 1240, and has a volume displacement member-type pump 1230 (e.g., balloon) being inflated to expand within the pump body 1221. As the pump 1230 inflates, it displaces surrounding fluid, thereby pushing fluid both distally toward the inflow region 1220i and proximally toward the outflow region 1220b. Fluidic pressure causes the inlet valve 1240 to close, while also urging fluid to exit the pump body 1221. As shown in FIG. 1C, when the inlet valve 1240 is fully closed, all of the fluid volume in the pump body 1221 exits through the outlet of the outflow region 1220b.

Inflation of the pump 1230 also helps generate momentum of the fluid column traveling toward the outflow region 1220b in the pump body 1221. FIG. 1D illustrates when the pump 1230 is at an end portion of the expansion phase, and the pump 1230 is inflated to a maximum volume. At this stage of operation, the fluid mass in the pump body 1221 has momentum toward the outflow region 1220b to exit the pump body 1221 through the outlet, and such movement of the fluid mass results in a negative pressure within the pump body 1221. Under such momentum and negative pressure within the pump body 1221, the inlet valve 1240 opens and additional fluid is pulled into the pump body 1221 through the open inlet valve 1240 in the axial flow direction, as shown in FIG. 1D.

As shown in FIG. 1E, when the pump 1230 enters its contraction phase and begins to deflate, the fluid momentum continues, and additional fluid is pulled into the pump body 1221 in the axial flow direction through the open inlet valve 1240. In some instances, the amount of fluid momentum may decrease at this stage if additional fluid is also pulled into the pump body 1221 through the outflow region 1220b. However, in these instances, the momentum (and volume) of fluid pulled through the inflow region 1220i is greater than that of fluid pulled distally through the outflow region 1220b. Accordingly, fluid momentum may slow, but still continues in the direction from the inflow region 1220i toward the outflow region 1220b, thereby drawing in additional fluid into the pump body 1221 for further pumping.

FIG. 1F illustrates when the pump 1230 is at an end portion of the contraction phase, and the pump 1230 is deflated to a minimum volume. At this stage of operation, the fluid mass continues to have momentum in the proximal direction toward the outflow region 120b, and fluid continues to exit the pump body 1221 through the outlet of the conduit. Following the contraction phase, the pump 1230 returns to its expansion phase, and the above-described cycle of expansion and contraction (with continued momentum and fluid conveyance through the conduit outlet, as shown in FIGS. 1B-1F) may repeat. Further details regarding axial flow in the circulatory assist device 1200 are described in International Patent Application No. PCT/IB2024/057381, which is incorporated herein in its entirety by reference.

FIG. 1A illustrates an example circulatory assist device that may be used in conjunction with the docking station described herein. However, it should be understood that the docking station in accordance with the present technology can be configured to receive any suitable kind of circulatory assist device, including impeller-based circulatory assist devices, intra-aortic balloon pumps, as well as PCI devices, and/or other associated cardiac devices such as embolic filters. The docking stations in accordance with the present technology may further be configured to receive a plurality of such devices simultaneously, such as a circulatory assist device and a PCI device and/or embolic filter device through the same or separate channels in the docking station. Other examples of circulatory assist devices that may be used in conjunction with the docking station in accordance with the present technology are described in detail in U.S. patent application Ser. Nos. 18/300,207, 18/500,906, and International Patent Application No. PCT/EP2023/080613, each of which is incorporated herein in its entirety by this reference.

### I. Circulatory Assist Systems

In some variations, a circulatory assist system includes an aortic pump docking station (also referred to herein as an “docking station” that is positionable in a patient (e.g., in a cardiovascular vessel lumen, such as the aorta). For example, the aortic pump docking station may be positionable in a descending aorta of a patient. The docking station may function to receive and be operable with a circulatory assist device such as a percutaneous ventricular assist device (pVAD), though other circulatory assist devices for use in a cardiovascular lumen are contemplated.

In some variations, a medical device (e.g., docking station) may include an expandable support with an interior region and a seal arranged in the interior region. The expandable support may have a delivery configuration with a first diameter and a deployed configuration with a second variable diameter larger than the first diameter. The seal may include a seal opening and a sealing element configured to seal around a circulatory assist device seated in the seal opening. In some examples, the sealing element may comprise one or more flexible leaflets adapted to conform to and form a seal with an exterior surface of the circulatory assist device. In some variations, a seal between the circulatory assist device and the expandable support may additionally or alternatively be formed at least in part with a sealing element on the circulatory assist device. Furthermore, in some variations, a seal between the circulatory assist device and the expandable device may additionally or alternatively be formed with a sealing element configured to couple to the expandable device and/or circulatory assist device (e.g., a sealing element such as a balloon that is delivered separately from the expandable device and circulatory assist device).

In some variations, the docking station may be delivered to an aorta (e.g., descending aorta) of a patient and configured to receive (e.g., removably receive) a circulatory assist device, thereby helping to position the circulatory assist device in the aorta. The option of aortic placement of a circulatory assist device may be advantageous for a number of reasons. For example, placement of a circulatory assist device in the aorta is generally low invasive and/or results in low trauma, which may lower the risk of complications such as myocardial or cerebral infarction, and/or arrythmia that may result from interaction between the circulatory assist device and myocardium. As another example, placement of a circulatory assist device in the aorta may avoid interaction with the aortic valve, thereby reducing the potential for aortic valve injury that may result from a circulatory assist device being placed in a left ventricle of the patient. Furthermore, compared to placing a circulatory assist device in other cardiovascular regions such as the left ventricle, a procedure for placing a circulatory assist device in the aorta may be faster, simpler, and easier to perform. For example, the procedure may be simple enough for a clinician to perform using a skill level similar to that required to place an arterial line. As another example, the procedure to place a circulatory assist device in an aorta may be performed without imaging guidance, such that it can be performed in emergency situations (e.g., ambulance) in which X-ray or other imaging guidance may not be available.

However, in many instances, a circulatory assist device placed in the aorta may have limited effectiveness in providing cardiovascular support, due at least in part by potential backflow or regurgitation around the device (e.g., from downstream to upstream back toward the aortic arch, etc.).

The aortic pump docking station in accordance with the present technology addresses various limitations of current circulatory assist devices. For example, the docking station may function to streamline and facilitate an easy-to-perform method of placing a circulatory assist device in an aorta, lessening the operator skill and/or equipment requirements that may otherwise be associated with circulatory support device placement. This may help contribute to faster patient treatment, as well as treatment with fewer complications. Furthermore, when deployed in a descending aorta of a patient and hosting a circulatory assist device, the docking station may function to help prevent backflow or regurgitation around the circulatory assist device to thereby improve the treatment effectiveness of the circulatory assist device. Advantageously, as further described herein, the docking station may be configured to be placed in a variety of anatomical environments (e.g., due to patients' anatomical variance) and accommodate a variety of sizes and/or shapes of circulatory assist devices.

Furthermore, as further described herein, the docking station advantageously allows an option for escalating treatment by repositioning the circulatory assist device that is operable with the docking station. For example, a circulatory assist device may additionally or alternatively be placed in a cardiovascular lumen other than the aorta, and repositioned among two or more different cardiovascular locations to provide different levels of cardiovascular support (e.g., treatment intensity). For example, the circulatory assist device may be initially placed in the descending aorta (e.g., seated in the docking station) to provide a first level of cardiovascular support, then repositioned to a transvalvular location in which the circulatory assist device is at least partially positioned in the left ventricle to provide a second level of cardiovascular support or treatment intensity greater than the first level of cardiovascular support or treatment intensity. When less cardiovascular support is needed by the patient, the circulatory assist device may then be repositioned again in the descending aorta (e.g., seated in the docking station) to tune or adjust the provided cardiovascular support downwards, such as to wean the patient off cardiovascular support. However, in some variations the circulatory assist device may initially be at least partially placed in a left ventricle (and optionally, subsequently repositioned in the descending aorta to tune or adjust the provided cardiovascular support downwards, similar to that described above). In some variations, the docking station alone (that is, without the circulatory assist device seated therein) may have limited to no interference with native aortic flow, such that the docking station may remain in the aorta for at least some time even while the circulatory assist device is not seated in the docking station. Accordingly, the docking station may remain in the patient until its use with the circulatory assist device is desired, such as for weaning purposes. However, in some variations the docking station may remain implanted in the patient (e.g., permanently). In some of these variations, the docking station may be repositioned or otherwise adjusted to reduce the amount of cardiovascular support provided by the circulatory assist device on a long-term basis.

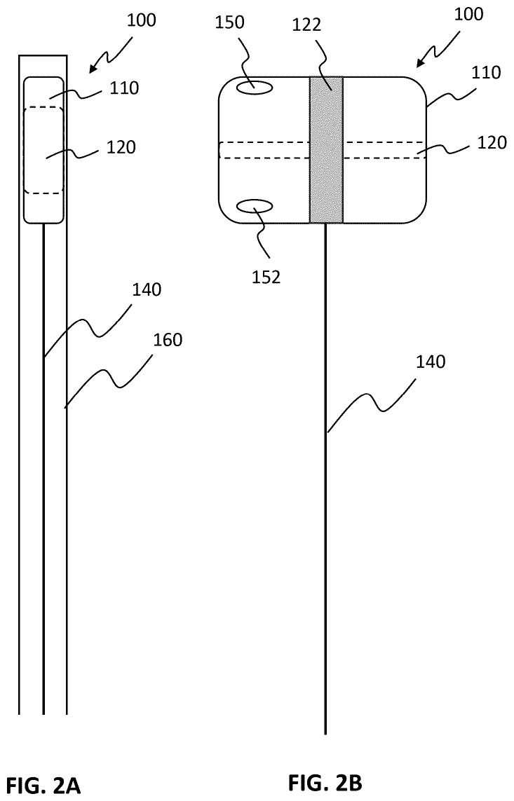

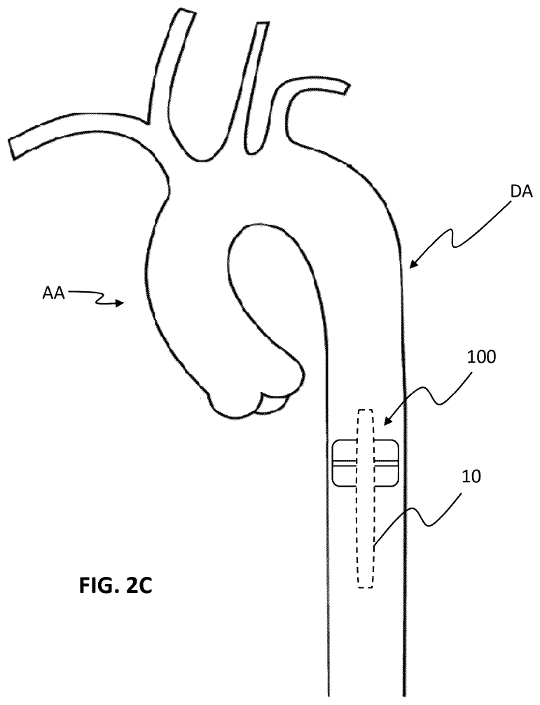

For example, FIGS. 2A and 2B are schematic illustrations of an example aortic pump docking station 100 operable with a circulatory assist device (e.g., pVAD). The docking station 100 may include an expandable support 110 with an interior region, and a seal 120 arranged within the interior region of the expandable support 110. The expandable support 110 may be coupled to an elongate member 140, which in some variations may assist with the placement of the support 110 at a treatment site.

As shown in FIG. 2A, the expandable support 110 may have a radially collapsed delivery configuration to allow the expandable support 110 and seal 120 to be held in a sheath 160. When the expandable support 110 is in this delivery configuration in the sheath 160, the docking station 100 may, for example, be configured for percutaneous delivery and navigation to a treatment site in a descending aorta. In some variations, proximal retraction of the sheath 160 (and/or distal advancement of the support 110) may expose the support 110 and allow the support 110 to radially expand to a deployed configuration (FIG. 2B).

The seal 120 may have a seal opening 122 configured to axially receive a circulatory assist device (not shown), thereby allowing the circulatory assist device to “dock” to the support 110. When the circulatory assist device is received in the seal opening 122 and the support 110 is placed in a cardiovascular lumen (e.g., aorta), the circulatory assist device may be operated to provide cardiovascular support to the patient. For example, as shown in FIG. 2C, the docking station may be positioned in a descending aorta (DA) after advancing the docking station 100 from a percutaneous puncture site (e.g., insertion site at a femoral artery). In some variations, a circulatory assist device 10 may be positioned in the seal opening of the docking station 100 after the docking station 100 is deployed at the treatment site. However, in other variations, a circulatory assist device 10 may be positioned in the seal opening of the docking station 100 prior to deployment of the docking station 100 at the treatment site. For example, the circulatory assist device 10 may be delivered together with the docking station 100 to the treatment site, and the docking station 100 may subsequently be expanded to the deployed configuration while the circulatory assist device 10 is received in the docking station 100. Furthermore, in some variations, a circulatory assist device 10 may be deployed after a first portion of the docking station (e.g., expandable support 110) is positioned at the treatment site, and after a second portion of the docking station (e.g., the seal 120 and/or other sealing element) is separately positioned at the treatment site. For example, the expandable support 110 may be delivered and expanded to its deployed configuration in a first delivery procedure (e.g., via a first catheter), and the seal 120 and/or other sealing element (e.g., inflatable member, etc.) may be delivered, optionally coupled to the expandable support 110, and expanded to its deployed configuration. When the docking station 100 is delivered and deployed, it can receive the circulatory assist device 10 as shown in FIG. 2C.

Further details of various features and variations of the docking station are described below.

### A. Support

The support 110 functions at least in part to provide structural support for the seal 120 and form a peripheral seal between the docking station 100 and an inner wall of the cardiovascular lumen (e.g., descending aorta). Furthermore, in some variations the support 110 functions to narrow a functional inner diameter of the aorta, in order to help make sealing around the circulatory assist device (e.g., by adjusting to different diameters of the aorta) more feasible.

In some variations, the support 110 may be configured to conform to an inner wall of a cardiovascular lumen and form a substantially fluid tight seal between the docking station 100 and the lumen wall. This seal may, for example, help reduce or prevent backflow or regurgitation of flow back toward the heart. Furthermore, since size and/or shape of a cardiovascular lumen may vary depending on individual patient anatomy, the support 110 may also be configured to have a variable diameter in the deployed configuration to accommodate different sizes and/or shapes of lumen walls at the treatment site.

Accordingly, in some variations, the support 110 may be expandable and/or include a flexible, compliant outer surface. For example, the support 110 may include an expandable structure including a fluid impermeable membrane (e.g., polymer membrane). For example, the support 110 may include an expandable frame or other support adjacent to the membrane, and the expandable frame may be configured to urge the membrane against a lumen wall to form a peripheral seal with the lumen wall. For example, the support 110 may include an expandable frame such as a stent or mesh that is configured to self-expand (e.g., the frame may include shape memory material such as nitinol) and/or be expanded with a separate device such as a balloon device.

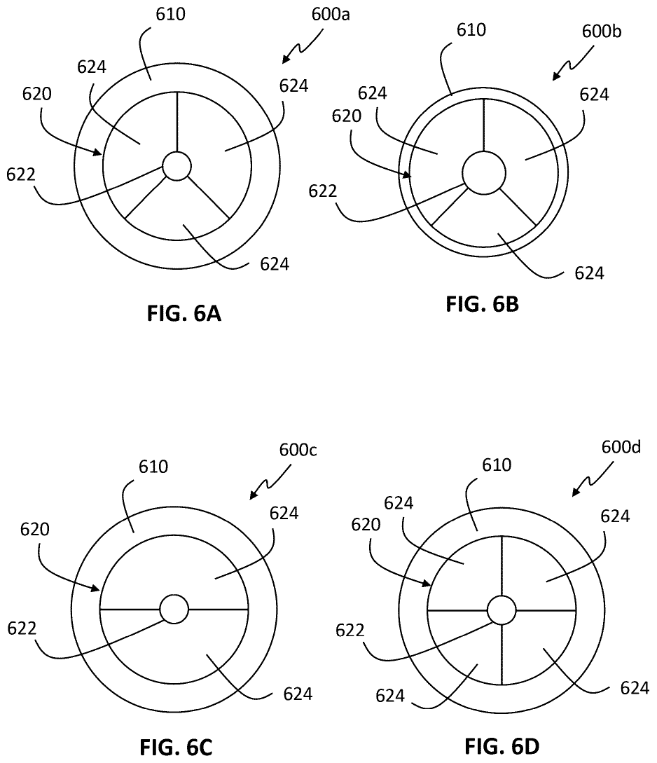

As another example, in some variations, the support 110 may include an inflatable member including the membrane, such that inflation of the inflatable member can urge the membrane against a lumen wall to form a peripheral seal with the lumen wall. In these variations, the amount of inflation (and corresponding amount of expansion) of the support 110 may enable the support 110 to conform to a range of different cardiovascular lumen sizes and/or shapes. For example, FIGS. 6A and 6B are schematic illustrations of example docking stations 600a and 600b. Docking stations 600a and 600b are shown as including an expandable support 610, which may for purposes of this discussion include an inflatable member. The support 610 in FIG. 6A has a larger outer diameter in an expanded state, which may be attributed to a greater amount of inflation, while the support 610 in FIG. 6B has a smaller outer diameter, which may be attributed to lesser amount of inflation. Accordingly, the support 610 in FIG. 6A may be configured for sealing against a larger lumen, while the support 610 in FIG. 6B may be configured for sealing against a smaller lumen. In some variations, the inflatable member may include various discrete points of contact against the lumen wall, to help accommodate a range of outer diameters for sealing purposes. For example, the support 110 may include an inflatable member having a corrugated balloon (e.g., wavy outer surface), inflatable circumferential and/or longitudinal ribs, and/or the like. Additionally or alternatively, in some variations, the support 110 may include an expandable support similar to support 610, where the expandable support additionally functions to provide and/or enhance a peripheral seal around a circulatory assist system 10 (e.g., to replace or operate in combination with the seal 120). For example, the support 110 may be configured to provide an outer seal against the vessel lumen wall, as well as an inner seal against a circulatory assist device 10 seated within the interior region of the support 110. In some variations, the support 110 may include an inflatable member (e.g., helical or toroidal balloon) that provides both such outer and inner seals. However, in some variations the support 110 may include multiple inflatable members, including at least one inflatable member that provides the outer seal, and at least one inflatable member that provides the inner seal. For example, the support 110 may include at least one outer inflatable member mounted on an outer surface of an expandable structure (e.g., frame) and at least one inner inflatable member mounted on an inner surface of the expandable structure.

In some variations, the support 110 may engage with the cardiovascular lumen in an atraumatic manner (e.g., without anchoring). Varying pressure gradients within the cardiovascular lumen during device operation may urge the circulatory assist device 10 to move (e.g., longitudinally, rotationally, etc.), so engagement with the cardiovascular lumen may be advantageous in some instances to help avoid trauma to surrounding tissue. Accordingly, the support 110 may include a soft, flexible material with a smooth peripheral edge and smooth surface(s). Furthermore, the support 110 may omit anchoring features (e.g., tines, hooks, etc.) along the peripheral edge and/or on the support surface.

In some variations, the support 110 may have a generally round cross-section and a vacant interior region in which the seal 120 may be arranged. For example, as shown in FIG. 2B, in some variations the support 110 may be generally ring-shaped or tubular (e.g., band, toroid, sleeve, cuff, etc.).

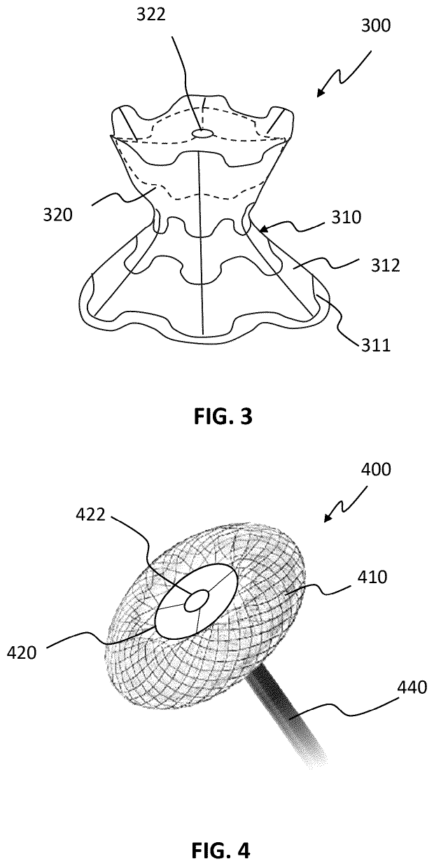

FIGS. 3-5C are schematic illustrations of example expandable supports for an aortic pump docking station. Various features of these examples may be combined in any suitable manner.

FIG. 3 depicts an example docking station 300 with an expandable support 310 including a flexible stent frame 311 and at least one membrane 312 coupled to or adjacent to the stent frame 311. The stent frame 311 may include one or more struts and/or include a suitable mesh structure including wires or a laser-cut material, such as nitinol or other shape memory material such that the stent frame 311 is self-expandable. While FIG. 3 depicts the stent frame 311 as having a particular strut pattern, it should be understood that other variations may include other strut or mesh patterns (e.g., interwoven mesh, mesh with diamond-shaped cells, one or more wound helical filaments, etc.). In some variations, the expandable support 310 may include an outer membrane coupled to or adjacent to an outer surface of the stent frame 311 and/or an inner membrane coupled to an inner surface of the stent frame 311. The stent frame 311 has an inverted skirt or hourglass shape, with a narrower central region that provides a functional aortic lumen with a reduced diameter. Furthermore, outwardly flared profile of the stent frame 311 arranged distal to and/or proximal to the narrower central region may help facilitate the expandable support 310 to be suitable sized for lumen walls of varying sizes and/or shapes. As shown in FIG. 3, the docking station 300 may further include a seal 320 arranged in an interior region of the expandable support 310. For example, the plane of the seal 320 may be located distal to the narrowed central region of the stent frame 311, proximal to the narrowed central region of the stent frame 311, or substantially coincident with the narrowed central region of the stent frame 311.

FIG. 4 depicts an example docking station 400 with an expandable support 410 including a flexible toroidal stent frame. The stent frame may include one or more structs and/or include a suitable mesh structure including wires or a laser-cut material, such as nitinol or other shape memory material such that the stent frame is self-expandable. Furthermore, while FIG. 4 depicts the stent frame as having a particular mesh pattern, it should be understood that other variations may include other mesh patterns (e.g., interwoven mesh, mesh with diamond-shaped cells, etc.). In some variations, the expandable support 410 may include at least one membrane similar to that described above with respect to FIG. 3. The outer diameter of the toroidal stent frame may be variable (e.g., depending on the amount of size of lumen in which the docking station 400 is placed) to accommodate lumen walls of varying sizes and/or shapes. As shown in FIG. 4, the docking station 400 may further include a seal 420 arranged in a central region of the toroidal expandable support 410.

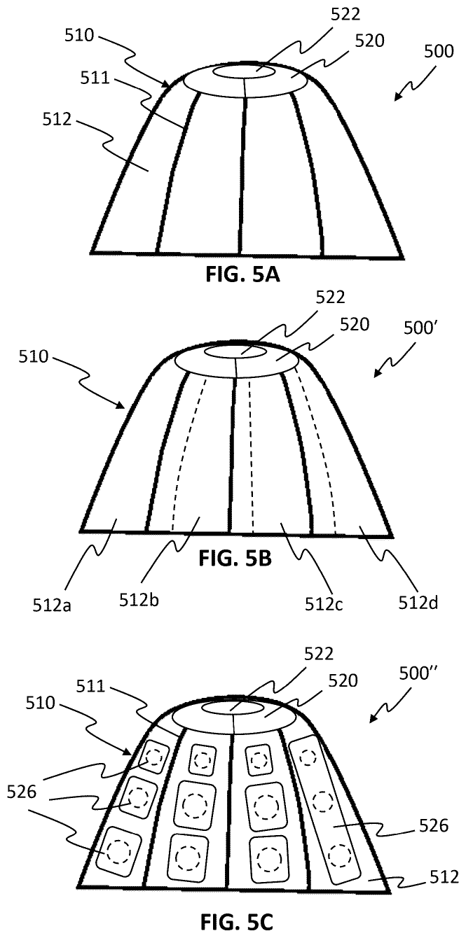

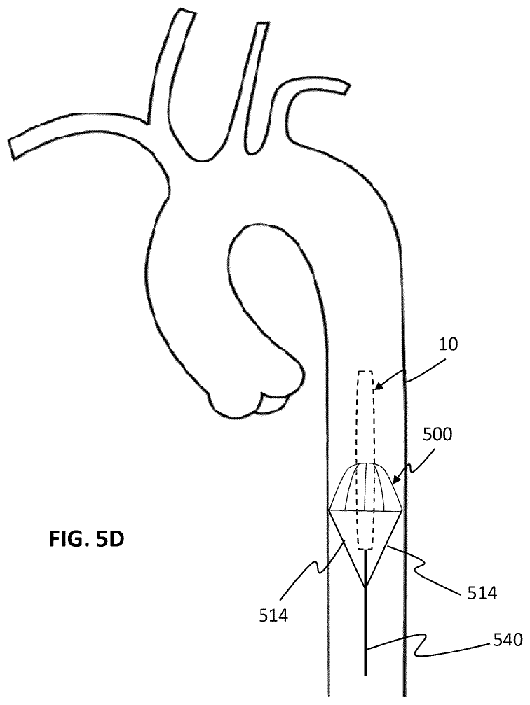

In some variations, a docking station may include an outwardly radially flared support with a closed state in which the support forms a peripheral seal against the lumen wall, and an open state in which the support does not form such a peripheral seal. For example, FIG. 5A illustrates an example docking station 500 including an expandable support 510 having a generally outwardly flared shape (e.g., dome, umbrella, skirt, cone, cup, and/or the like) with a narrower first end and a peripherally open second end opposite the first end. For example, in some variations the narrower end of the expandable support 510 may be an upstream or distal end, and the peripherally open end may be a downstream or proximal end. However, in some variations the narrower end of the expandable support 510 may a downstream or proximal end, and the peripherally open end may be an upstream or distal end. The docking station 500 may further include a seal 520 located at the narrower distal end, where the seal 520 includes a central seal opening 522 (e.g., defined in leaflets, as further described below with respect to FIGS. 6A-6D). The support 510 may be radially expanded in a closed state, and radially contracted in the open state. To help prevent inversion of the support 510 (e.g., due to pressure gradient), in some variations the support 510 may include a frame with one or more reinforcement members 511. Reinforcement members 511 may comprise a resilient, flexible material (e.g. an elastic or superelastic metal such as Nitinol) having sufficient rigidity to maintain the deployed shape of the support 510, yet being collapsible into a radially compact configuration for delivery. The reinforcement members 511 may be radially distributed (e.g., similar to umbrella arms), arranged in a pattern of interconnecting struts, or in a woven mesh, to form a support structure for a membrane 512. In some variations, the membrane 512 may include a sheet of material extending peripherally around the support 510. Additionally or alternatively, the membrane 512 may include multiple radial segments of material interspersed between adjacent reinforcement members 511 (and may be coupled to one or more reinforcement members 511, such as by suturing).

Additionally or alternatively, the docking station may include one or more attachment members 514 (as shown in FIG. 5D) coupling a proximal portion of the support 510 to the elongate member 540. Further details regarding the attachment members 514 are described below.

When in the closed state, the support 510 may substantially prevent, inhibit, or partially limit backflow of fluid in a proximal-to-distal direction toward the heart. In some variations, the support 510 may be in this closed state when pressure on a proximal (downstream) side of the support 510 is greater than the distal (upstream) side of the support 510. The support 510 may also be in this closed state when pressure on the proximal (downstream) side of the support 510 is about equal to the distal (upstream) side of the support 510. Closure of the support 510 may help to maintain a pressure gradient in the descending aorta that is created by the circulatory assist device. For example, when the support 510 is closed, it may maintain a higher pressure in the proximal side of the support 510 and in the peripheral system, thereby maintaining organ perfusion, especially kidney perfusion. This arrangement also helps to maintain a lower pressure on the distal side of the support 510, thereby advantageously reducing the workload for the heart.

Furthermore, in some variations, the support 510 may be configured to gradually shape the outflow of fluid from the circulatory assist device 10 from a narrower, more focal profile to a wider, more divergent profile. This widening or diverging of the outflow of fluid may help reduce the exit speed of the pumped fluid, thereby reducing the likelihood of turbulent flow that may cause complications such as hemolysis.

When the circulatory assist device 100 is deployed at a treatment site, the support 510 may toggle between its closed state and open state at least in part passively in response to pressure differential across the support 510 as described above, and/or may toggle between its closed state and open state at least in part actively in response to proximal or distal actuation of the attachment members 514.

FIG. 5B is a schematic illustration of an example docking station 500′ that may be similar to the docking station 500 described above with respect to FIG. 5A, except as described below. For example, the docking station 500′ may include a support 510 with multiple leaflets or segments of membrane material arranged in an overlapping manner. For example, at least four leaflets 512a, 512b, 512c, and 512d may be overlapped or nested in series around the periphery of the support 510. In some variations, the amount of overlap between leaflets may generally correspond to the amount of radial contraction or radial expansion of the support 510. For example, when the support 510 is in the open state, the leaflets may be less overlapped than when the support 510 is in the closed state.

In some variations, a support 510 may maintain a peripheral seal against the inner wall of the cardiovascular lumen during both the closed and open states of the support, and a membrane of the support may include one or more additional sub-valves that prevent or permit the passage of fluid through the support in the closed or open states, respectively. For example, FIG. 5C is a schematic illustration of an example docking station 500″ including a membrane 512 (e.g., similar to that described above with respect to FIG. 5A) including one or more flap valves 526. A flap valve 526 may, for example, include at least one aperture and at least one flap. A particular flap valve 526 may have one aperture and one flap, multiple apertures and one flap, or one aperture and multiple flaps. Pressure differential between upstream and downstream sides of the docking station 500″ may, for example, cause movement of the flap(s) relative to the aperture(s) for each flap valve 526. In some variations, flap valves for the docking station may be similar to those described in greater detail in International Patent Application No. PCT/EP2023/059293, which is incorporated above by reference.

As shown in FIGS. 2A and 2B, in some variations the docking station 100 may further include an elongate member 140 to which the support 110 is attached. The elongate member 140 may, for example, function as a delivery member to help guide the positioning (e.g., advancement, repositioning, retrieval, etc.) of the support 110 in a cardiovascular lumen. Additionally or alternatively, the elongate member 140 may include one or more lumens associated with controlling feature(s) of the support 110. For example, the elongate member 140 may include one or more lumens for an inflation fluid configured to expand an inflatable variation of the support 110, pull wires or other attachment members for expanding and/or contracting the support 110, and/or the like. In some variations, the elongate member may be removably coupled to the support 110, such as with a temporary connection. For example, a distal end of the elongate member 140 may be connected to a proximal portion of the support 110 by a mechanical fastener, perforation, magnets, snare and/or the like, such that the elongate member 140 may be decoupled from the support 110 by disengaging a mechanical fastener, breaking a perforation with sufficient proximal or twisting force, etc. In some instances, as further described herein, the elongate member 140 may decouple from the support 110 after delivering the support 110 to a treatment site, so as to allow the support 110 to remain at the treatment site without delivery components and/or other components of the cardiovascular assist system.

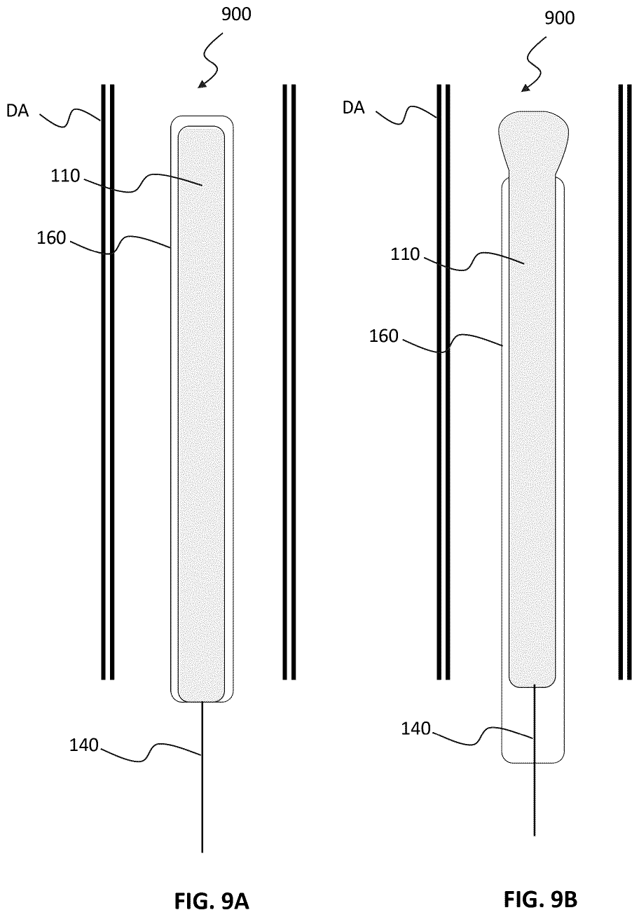

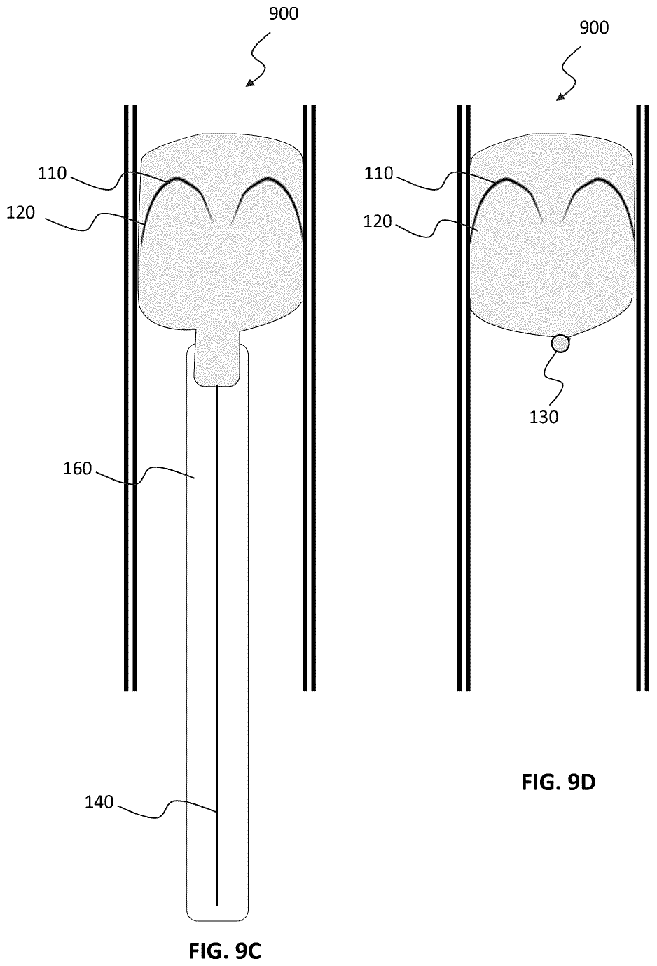

In some variations, the docking station 100 may further include one or more engagement features configured to aid retrieval and/or repositioning of the support 110 from a treatment site, for example when the elongate member 140 has been decoupled from the support 110. For example, as shown in FIG. 9D, the support 110 may include an engagement feature 130 at a proximal portion of the support 110. The engagement feature 130 may, for example, include a knob, a hook, and/or other feature configured to be engaged by a retrieval device (not shown) such as a grasper. Engagement between the engagement feature 130 and the retrieval device may facilitate radial contraction and/or retrieval of the support 110 (e.g., via proximal withdrawal of the retrieval device).

In some variations, the docking station 100 may be configured such that it is not retrieved and/or repositioned. For example, in some variations at least a portion of the docking station 100 (e.g., support 110) may include a bioabsorbable material (e.g., any one or more of magnesium, polyglycolides and their copolymers, polylactides and their copolymers, etc.) so as to naturally removed from the patient without active retrieval of the full docking station as described above. As another example, in some variations at least a portion of the docking station 100 may be reconfigured for long-term placement in the patient, such as adjusted to provide at least partial blood flow restoration (e.g., after the removal of the circulatory assist device 10). For example, following removal of the circulatory assist device 10, the seal 120 may be in a sufficiently open state to allow for blood flow through the seal opening in the absence of the circulatory assist device 10. Additionally or alternatively, in some variations the support 110 may be configured to transition to a flow restoration configuration in which a diameter of the interior region is larger than the seal opening. Additionally or alternatively, the docking station 100 may be repositioned to a different location in the vessel where it the docking station 100 is not actively operated in conjunction with a circulatory assist device.

### B. Seal

As described herein, the seal 120 may be arranged within an interior region of the support 110, and functions to form a substantially fluid tight seal around the circulatory assist device 10 to help prevent backflow of pumped fluid.

In some variations, the seal 120 may include a flexible membrane material, such as a flexible polymer material. The seal 120 may be coupled to the support 110 with one or more fastening elements, such as sutures. For example, the seal 120 may be coupled to an inner surface of the support 110 around a periphery of the seal 120. In some variations, the seal 120 may be substantially coaxial with the support 110 (e.g., centered within the interior region of the support 110), or may be radially offset from a center of the support 110.

As described herein, the seal 120 may include a seal opening 122 shaped and sized to receive the cross-section of a circulatory assist device 10, thereby allowing the circulatory assist device 10 to be seated in the seal opening 122. In some variations, the seal opening 122 is generally radially centered within the seal 120 (e.g., such that the circulatory assist device 10 is generally centered within the seal 120 and/or support 110), but in some variations the seal opening 122 may be radially offset from a center of the seal 120 (e.g., such that the circulatory assist device 10 is radially off-center within the seal 120 and/or support 110). The seal 120 may further include one or more sealing elements configured to seal around the circulatory assist device 10 when the device 10 is seated in the seal opening 122. In some variations, to accommodate a variety of cross-sectional sizes and/or shapes of circulatory assist devices, the seal opening 122 may have a dynamic or variable size and/or shape. For example, the seal opening 122 may have a dynamic or variable diameter that adjusts to the size and/or shape of a variety of circulatory assist devices. The variable diameter may, for example, be accomplished by adjustments in the one or more sealing elements (e.g., shape). Additionally or alternatively, different seal openings may be sized for different circulatory assist devices (e.g., docking stations with larger seal openings may be intended for use with larger circulatory assist devices).

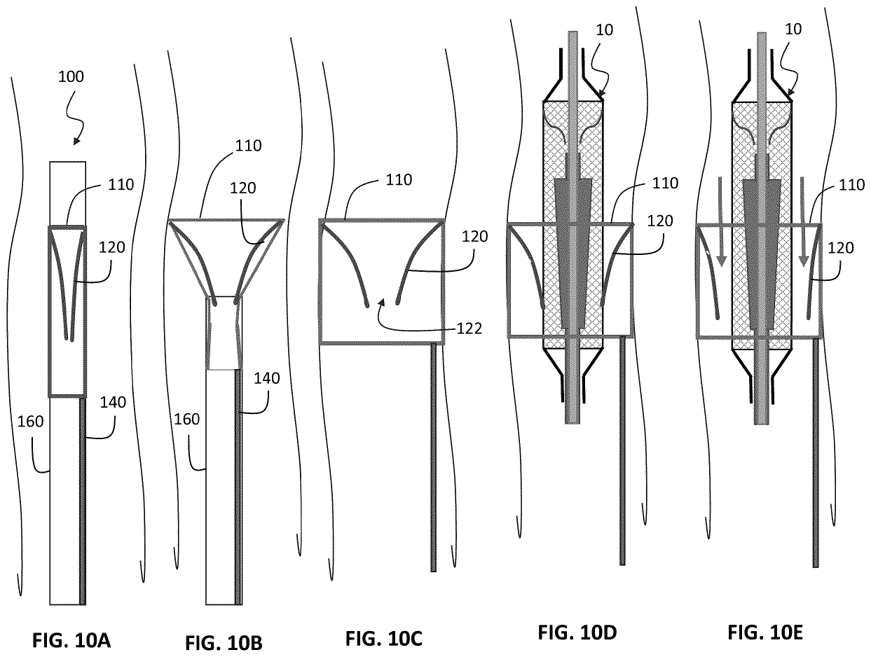

FIGS. 10A-10E show schematic illustrations of an example docking station 100 having a seal 120 with such a dynamic seal opening 122. As shown in FIG. 10A, the seal 120 may have a seal opening 122 with a smaller inner diameter when the docking station 100 is in a delivery configuration. As shown in FIGS. 10B and 10C, the seal opening 122 may begin to transition to a larger inner diameter as the docking station transitions to the deployed configuration shown in FIG. 10C. The seal opening 122 as shown in FIG. 10C may be at a maximum resting diameter in the absence of a circulatory assist device 10 seated therewithin, and/or may be further adjustable to various diameters once the docking station 100 is deployed. For example, as shown in FIG. 10D, the seal opening 122 can expand to accommodate the outer diameter of a circulatory assist device 10 seated in the seal opening 122. The sealing element can form a peripheral seal around the circulatory assist device 10. However, in some variations, the seal opening 122 may increase as shown in FIG. 10E, thereby permitting blood flow to occur between the circulatory assist device 10 and the seal 120 through the seal opening 122. For example, if pressure upstream of the seal 120 is greater than pressure downstream of the seal 120, then the seal opening 122 may passively increase in size to have a diameter greater than the diameter of the portion of the circulatory assist device 10 seated in the seal 120.

The seal opening 122 may be defined by various seal features. In some variations, the seal 120 may include a sealing element comprising a plurality of leaflets. For example, FIG. 6A is a schematic illustration of an example docking stations 600a including a valve-type seal including a plurality of leaflets 624 configured to coapt against each other. The free ends of the leaflets 624 may include an arcuate cutout, such that when the leaflets 624 coapt against one another to substantially close the seal 120, the coapting leaflets may define a seal opening 622 that is generally circular, even in the absence of a circulatory assist device 10 therein. Accordingly, the periphery of the circular seal opening 622 may match or be complementary to the cross-sectional shape of the circulatory assist device 10 for enhanced sealing against backflow. Furthermore, the seal opening 624 may, in some instances, dynamically scale in size to accommodate larger diameter circulatory assist devices, in that the free arcuate ends of the leaflets 624 may curl up or down (e.g., as a lip or rim) to collectively define a larger circular seal opening 622. As shown in FIG. 6A, the seal opening 622 is generally centered within the seal 620, though in some variations the seal opening 622 may be radially offset from a center of the seal 620 and/or support 610.

As discussed above, in some variations, the seal opening may defined at least in part by cutout(s) in one or more seal membrane features. The seal opening may reduce the amount of seal (e.g., valve) material in the docking station and facilitate a delivery system that has a reduced cross-sectional profile (e.g., lower French size for a delivery sheath or catheter). Accordingly, the seal opening may furthermore be advantageous for reducing patient trauma when the docking station and/or circulatory assist device are being placed in the patient.

In some variations, the sealing element of a valve-type seal may include a set of leaflets configured to coapt against one another. For example, FIGS. 6A-6D depict example variations of docking stations 600a-600d, respectively, each including a plurality of leaflets 624. In some variations, a docking station may include two leaflets (e.g., as shown in FIG. 6C), three leaflets (e.g., as shown in FIGS. 6A and 6B), four leaflets (e.g., as shown in FIG. 6D), or more leaflets. For example, docking stations 600a and 600b may include tricuspid valves (with a central seal opening 622 formed by arcuate cutouts in the leaflets, as described above). In other variations, the sealing element of the seal may include one or more suitable coapting features. For example, in some variations, the seal may be a duckbill valve.

As another example, in some variations the seal 120 may additionally or alternatively include one or more expanding sealing elements. For example, the seal 120 may include one or more inflatable rings or other annular seals (e.g., toroidal balloon, circumferential rings or ribs, etc.) configured to surround the circulatory assist device and expand into sealing engagement with it. In such examples, the docking station may include an inflation tube in fluid connection with the inflatable seal and configured to extend through the vessel out of the patient for connection to an inflation device. As another example, the seal 120 may include a self-expanding element such a self-expanding foam (e.g., sponge-like material) or shape memory material (e.g., nitinol) to expand radially inwards and reduce the diameter of the seal opening 122. The expanding sealing elements may be configured to adjust the seal opening 122 smaller and/or larger, such as depending on the diameter of a circulatory assist device 10 to be seated within the seal opening 122.

In some variations, the opening in which the circulatory assist device 10 is seated may have a fixed or substantially fixed inner diameter that matches or complements the outer diameter of the circulatory assist device 10. For example, in the absence of a circulatory assist device 10 in the seal 120, the seal opening 122 may define a free or open passageway having a diameter that is substantially equal to the outer diameter of an anticipated circulatory assist device 10. This may reduce impedance to blood flow through the valve opening 122 when a device is not received in the docking station, yet provide a seal with the circulatory assist device when received therein. Further, the seal 120 may be shaped and otherwise configured to avoid interference with the circulatory assist device 10 and to resist deformation or inversion of seal 120 (or valve leaflets thereof, if present) when the circulatory assist device is being inserted therein. For example, the seal 120 may have leaflets with free downstream edges that curve or angle outwardly in an unbiased condition to facilitate receiving the circulatory assist device between the leaflets. Furthermore, the circulatory assist device may have a distal tip configured for insertion through seal 120, e.g. having a tapered tip configured to slide between the leaflets and gradually urge them apart as the circulatory assist device is inserted.

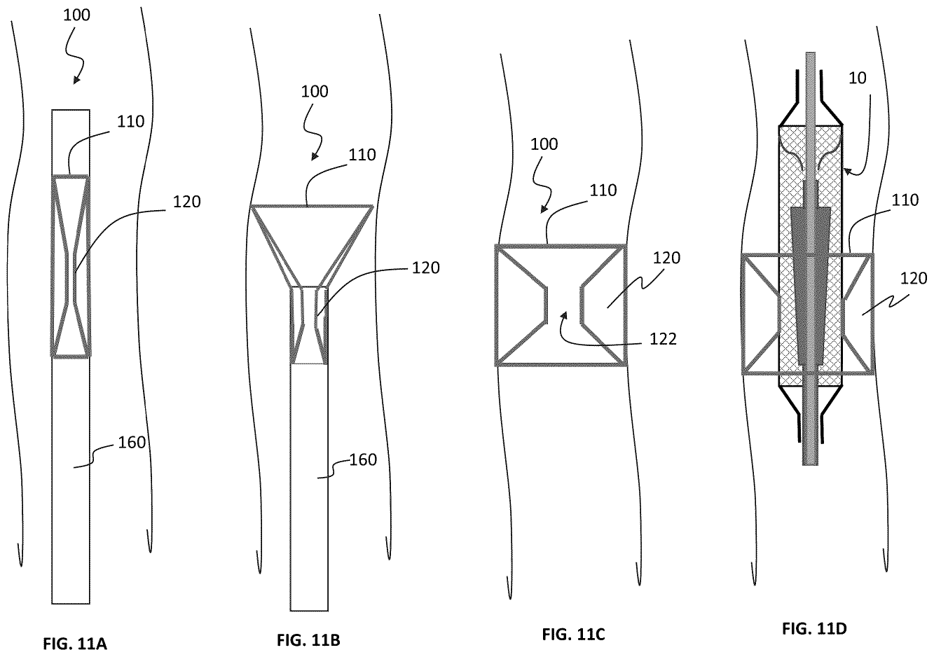

For example, in some variations the seal opening 122 may have a substantially static size that, in the absence of the circulatory assist device, leaves an opening or “hole” in the seal 120. FIGS. 11A-11D are schematic illustrations of the deployment of docking station 100 with such a fixed sized opening for receiving a circulatory assist device 10. As shown in FIG. 11A, the docking station 100 may be in a delivery configuration in which the support 110 is in a smaller, radially contracted state. As the docking station 100 is deployed, the support 110 transitions to a larger, radially expanded state as shown in FIGS. 11B and 11C. When in the deployed state, the seal 120 defines an opening 122 of a fixed diameter. As shown in FIG. 11D, a circulatory assist device 10 may be seated in the opening 122, such that the seal 120 forms a seal against the circulatory assist device 10. In some variations, one or more separate seals (e.g., similar to any of the seals 120 described here) may exist as part of the docking station 100 and/or circulatory assist device 10 that provides a secondary seal (e.g., against portions of the circulatory assist device 10 having different outer diameters than the diameter of the opening 122).

Although in many variations described herein, the seal 120 is described as part of the support 110 and/or part of the circulatory assist device 10, in some variations, the seal 120 may be deployed separately from the support 110, such as with a second catheter. Such separate elements may be coupled to or otherwise interact with the support 110 to form a seal around the circulatory assist device.

Additionally or alternatively, the sealing function of seal 120 may be performed instead or in supplementary fashion by one or more features on the circulatory assist device 10. In examples, the circulatory assist device may have an annular feature such as a rib or collar around its exterior which engages with a sealing element on docking station 110. Such feature may also serve to locate and limit the distal movement of the circulatory assist device relative to docking station 110 to assist in placement and prevent migration during use. Additionally or alternatively, in some examples, the outer surface of the circulatory assist device 10 may include a sealing element that can adjust to the inner diameter of the seal opening 122. The sealing element on the circulatory assist device 10 may, for example, include a member configured to expand (e.g., inflatable ring or rib, expanding foam, expanding umbrella or skirt). Additionally or alternatively, the circulatory assist device 10 may include a tapered profile of varying diameter, such that the circulatory assist device 10 may be seated within the seal opening 122 at a seating depth corresponding to where the diameter of the circulatory assist device 10 matches the diameter of the seal opening 122.

In some variations, the seal 120 may include one or more bioabsorbable materials, such that the seal 120 may transition to a sufficiently open state or flow restoration configuration in which blood can flow through the docking station (e.g., in variations in which the docking station is not removed through retrieval or otherwise, following removal of the circulatory assist device 10). For example, following removal of the circulatory assist device 10 from the docking station 100, the seal 120 (whether with dynamic diameter or fixed diameter) can transition over a period of time to enable substantially unimpeded blood flow through the seal opening 122. Suitable bioabsorbable materials include any one or more of magnesium, polyglycolides and their copolymers, polylactides and their copolymers, etc.

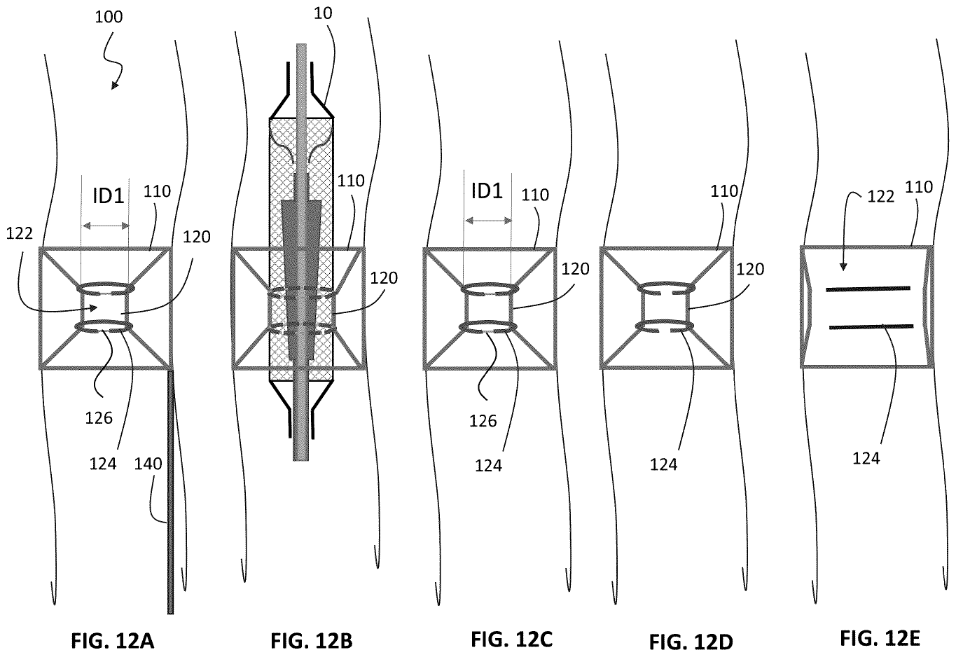

FIGS. 12A-12E are schematic illustrations of an example seal 120 with a bioabsorbable material. As shown in FIG. 12A, the seal 120 (shown in a deployed configuration with the support 110 expanded against a vessel wall) may include a tether 124 that regulates the diameter of the seal opening 122 (e.g., through tension). The tether 124 may, for example, be configured to constrain the diameter of the seal opening 122 and/or the interior region of the support 110 to a suitable diameter for sealing against a circulatory assist device 10, as shown in FIG. 12B. The tether 124 can be coupled to the support 110 (e.g., woven into or tied to the support 110, or coupled with one or more fasteners, etc.) and/or other suitable interior portion of the support 110. The tether 124 may include one or more bioabsorbable portions 126 that is configured to enable release of the tether 124 for allowing an expansion of the seal opening 122 and/or interior region of the support 110. For example, as shown in FIGS. 12A-12C, the tether 124 may include a bridge segment 126 including a bioabsorbable material. The bridge segment 126 may be resorbed over time and eventually disappear and/or disengage from the tether 124 (FIG. 12D). For example, the bridge segment 126 may become absent from the tether 124 over a period of time ranging from between about 1 day and about 1 week, between about 1 week to about 1 month, at least 2 months, at least 3 months, at least 6 months, or at least 1 year. The absence of the bridge segment 126 may occur after the circulatory assist device 10 is removed from the docking station 100. As shown in FIG. 12E, without the bridge segment 126, the tether 124 may relax and transition the seal 120 to a flow restoration configuration in which the seal opening 122 is expanded to allow blood flow therethrough (e.g., at a normal flow rate). Although FIGS. 12A-12E illustrate an example variation in which only a portion of the tether 124 is bioabsorbable, it should be understood that in some variations, the entire tether 124 may be bioabsorbable.

In some variations, the valve or sealing element may be configured to seal with an outlet tube of a circulatory assist device from which blood exits into the aorta, proximal to the pump head of such device. In some cases, the sealing element may be configured to seal with an outlet tube having a smaller diameter than that of the pump head. In such cases the circulatory assist device may be positioned such that blood exits the outlet tube proximally (downstream) of the sealing element or valve. In other examples, the valve or sealing element may be configured to seal with a shaft of the circulatory assist device, which contains, for example, an inflation lumen, a guidewire lumen, or a conduit for electrical wires or a drive shaft. In such examples, the valve or sealing element may be configured to seal with a shaft which has a substantially smaller diameter than both the pump head and the outlet tube.

It will be understood that some or any of the features and elements described herein in connection with the use of a circulatory assist device with docking station 110 may also be applied to other types of intravascular devices used with docking station 110, alone or in conjunction with a circulatory assist device. For example, intra-aortic balloon pumps, embolic filter devices, coronary interventional devices, and other intravascular devices may include any of the features described herein with respect to circulatory assist devices to allow such devices to be received in docking station 110 and to seal with a valve or other sealing element therein.

### C. Sensors

In some variations, the aortic pump docking station 110 may include one or more pressure sensors configured to measure the pressure gradient across the seal 120. For example, the docking station 110 may include a first pressure sensor configured to measure a pressure on an upstream (or distal) side of the seal 120, and a second pressure configured to measure a pressure on a downstream (or proximal) side of the seal 120. The pressure gradient across the seal, as measured by these pressure sensors, may be used to inform calibration of settings for the circulatory assist device 10 (e.g., pressure settings for the circulatory assist device 10) for improving pumping efficiency, etc.

As shown in FIG. 2B, for example, a first pressure sensor 150 may be coupled to or otherwise arranged on a distal portion of the support 120, so as to measure pressure on an upstream side of the seal 120. A second pressure sensor 152 may be coupled to or otherwise arranged on a proximal portion of the support 120, so as to measure pressure on a downstream side of the seal 120. In some variations, multiple pressure sensors may be coupled to the distal portion and/or proximal portion of the support 120 to help provide redundancy and/or averaging of pressure measurements.

Additionally or alternatively, in some variations one or more pressure sensors may be separate from the support 120. For example, one or more pressure sensors may be arranged on a distal surface of the seal 120 (e.g., distal surface of a valve leaflet or other valve membrane) and/or on a proximal surface of the seal 120 (e.g., proximal surface of a valve leaflet or other valve membrane). As another example, one or more pressure sensors may be arranged on the elongate member 140 distal and/or proximal to the seal 120. As another example, one or more pressure sensors may be arranged on leads that are not attached directly to the support 110 or the seal 120, but may pass through one or more lumens in the elongate member 140 and/or outer sheath 160 to extend to respective measurement locations distal and/or proximal to the seal 120.

### D. Multi-Port Docking Station

In some variations, a docking station may be configured to accommodate the docking and/or passage (e.g., delivery and/or retrieval) of multiple medical devices, including one or more circulatory assist devices (e.g. circulatory assist device 10, such as circulatory assist device 1200 shown in FIG. 1A). For example, a docking station may be configured to accommodate the docking of two or more medical devices (e.g., intravascular devices) within a support (e.g., support 110). As another example, a docking station may be configured to accommodate the docking of at least one medical device and the passage of at least one other medical device therethrough (e.g., intra-aortic balloon pump, embolic filter device, coronary interventional device such as a coronary stent or cardiac lead device, and other intravascular devices, etc.). Accordingly, such a docking station configured to accommodate the docking and/or passage of multiple medical devices may be used in percutaneous coronary interventions (PCI), and/or the like. In some variations, the docking station may include multiple ports, where each port includes a channel configured to receive at least one respective medical device for docking and/or passage.

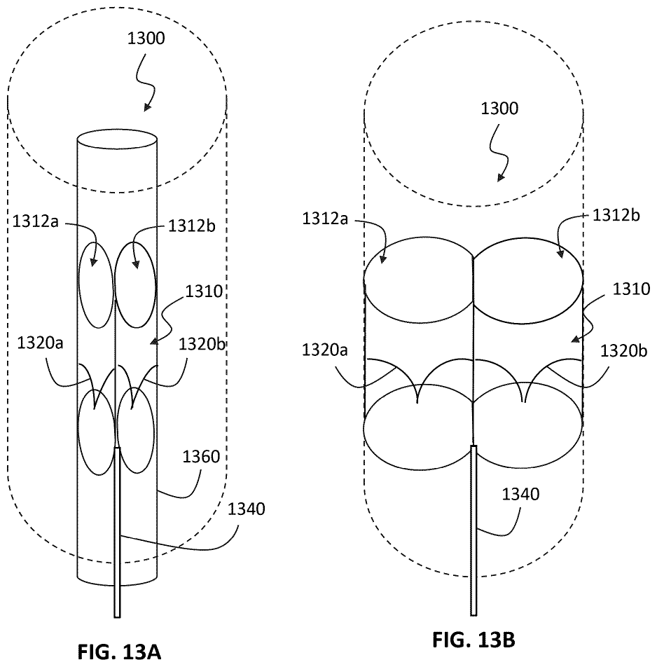

FIGS. 13A and 13B illustrate an example docking station 1300 (e.g., an example of docking station 100) including an expandable support 1310 (e.g., an example of support 110) and multiple ports extending axially through the docking station 1300. Although the docking station 1300 is shown as including two ports 1312a and 1312b, other examples may include three, four, or more ports. Each port may include a respective channel, which may have a constant or varying (e.g., tapering) diameter along the length of the docking station 1300. The channels of different ports may have the same diameter, inner and/or outer profile (e.g., cross-section), and/or other geometrical characteristics, or at least some of the channels of different ports may different diameter, profile and/or other characteristics. For example, the diameter of a first port 1312a may be larger than the diameter of a second port 1312b. As another example, the first port 1312a may have a first cross-sectional shape (e.g., circular) and the second port 1312b may have a second cross-sectional shape (e.g., oval) that is different than the first cross-sectional shape.

Like the docking station 100 as described elsewhere herein, the docking station 1300 may include at least one seal configured to form a seal around a medical device received therein. In the docking station 1300, each of the multiple ports may include a respective seal (e.g., seal 120 or other example seals described herein). For example, the first port 1312a may include a first seal 1320a, and the second port 1312b may include a second seal 1320b. Each of the seals 1320a and 1320b may be similar to any of the other example seals described herein (e.g., seal 120). For example, each of the seals 1320a and 1320b may include a valve, an inflatable member or other expandable member, etc. Furthermore, the seals in different ports may be generally the same or may be different. For example, in some variations both the first seal 1312a and the second seal 1312b may include valves, the first seal 1312a may include a valve and the second seal 1312b may include an inflatable member, or both the first seal 1312a and the second seal 1312b may include inflatable members.

Each of the multiple ports may be configured to receive a particular type (e.g., category, size, etc.) of medical device. For example, for any given port, the port size and/or other characteristics, the seal type, and/or the like may depend at least in part on the kind of medical device to be received through that port.

In operation, the docking station 1300 may be delivered to a treatment site in a vascular lumen for deployment. For example, as shown in FIG. 13A, the docking station 1300 may be delivered to a treatment site in a radially contracted state, such as within a sheath 1360 (e.g., an example of sheath 160 as further described herein) and with the aid of an elongate member 1340 (e.g., an example of sheath 140). The docking station 1300 may be deployed by releasing it from the sheath 1360 (e.g., through withdrawal of the sheath 1360 and/or advancement of the docking station 1300 from the sheath 1360), whereupon the support 1310 may expand to form a seal against the vascular lumen wall. The channels of ports 1320a and 1320b may also expand so that the docking station 1300 transitions to a deployed station (e.g., as shown in FIG. 13B). In the state shown in FIG. 13B, each port 1320a and 1320b may be configured to receive a respective medical device. For example, the first port 1320a may be configured to receive a circulatory assist device 10 for docking in the first port 1320a and providing circulatory assistance, while the second port 1320b may be configured to receive a second medical device (e.g., intravascular device, such as those described herein) and allow delivery of the second medical device to a second treatment location (e.g., heart). After treatment, the medical devices may be removed, and the docking station 1300 may be retrieved, transitioned to a flow restoration configuration, etc. similar to that described herein with respect to docking station 100. Further details of methods of operation of docking station 1300 are described below also with respect to docking station 100.

### II. Methods of Operation

Although the methods of operation are described herein primarily with reference to a medical device similar to the docking station 100, it should be understood that the methods described herein may additionally or alternatively be performed with any suitable variation of docking stations in accordance with the present technology.

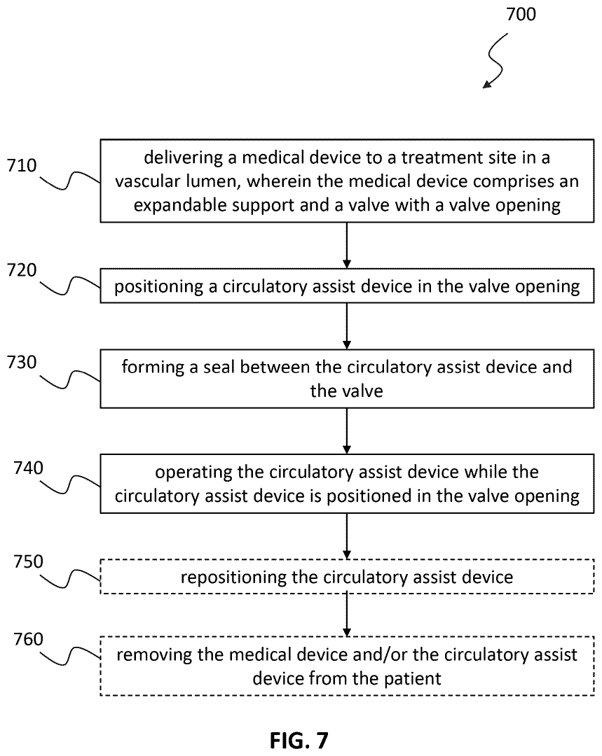

FIG. 7 is a schematic flowchart of an example method 700 of providing cardiovascular support to a patient. In some variations, the method 700 may include delivering a medical device to a treatment site in a vascular lumen 710, where the medical device includes an expandable support and a seal with a seal opening. The method may further include positioning a circulatory assist device in the seal opening 720, forming a seal between the circulatory assist device and the expandable support with the seal 730, and operating the circulatory assist device while the circulatory assist device is positioned in the seal opening 740. When treatment is no longer desired, the method may further include removing the medical device and/or the circulatory assist device from the patient 760.

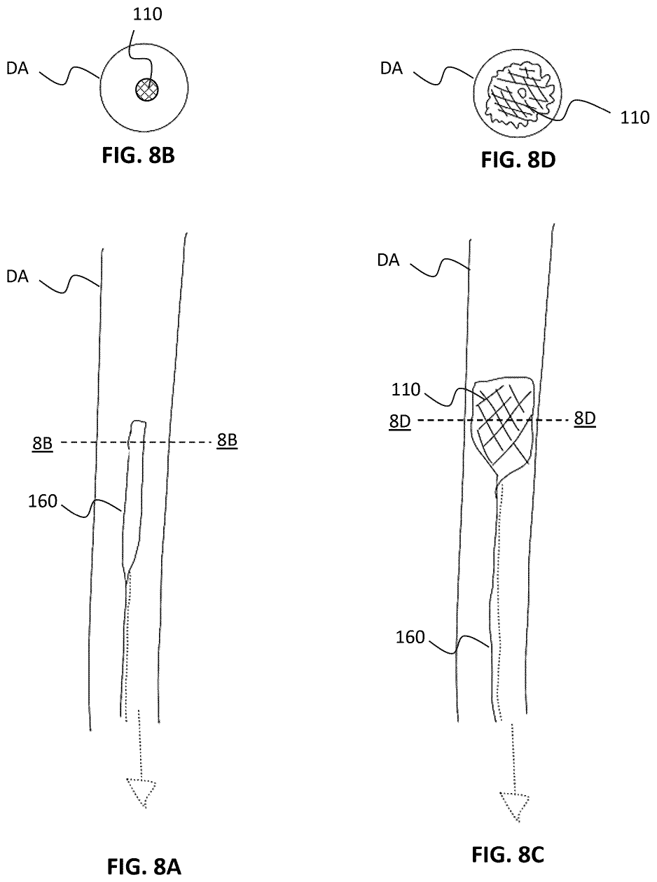

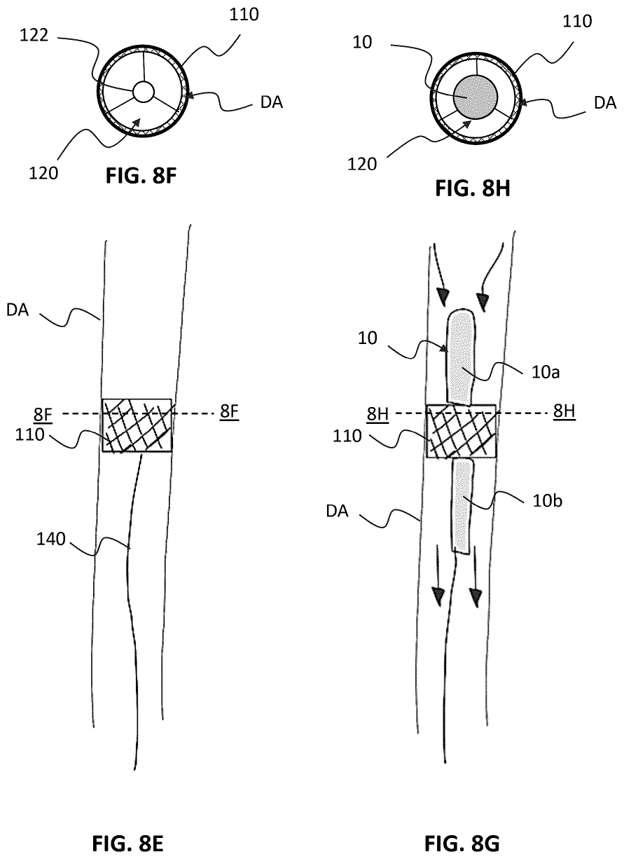

FIGS. 8A-8H illustrate various aspects of the method 700 performed with a docking station 100 as described herein. For example, as shown in FIG. 8A, delivering the docking station 100 to a treatment site in a vascular may include advancing a delivery system, including the outer sheath 160, into the descending aorta (DA). As shown in the cross-sectional view of FIG. 8B, the docking station 100 may be in a radially constrained state within the outer sheath 160 while being advanced to the descending aorta. In some variations, the delivery system with the docking station 100 may be introduced percutaneously into the patient trans-femorally at a femoral puncture site. However, in some variations the delivery system with the docking station 100 may be introduced into the patient through an upper access site (e.g., subclavian, axillary, radial, etc.).

As shown in FIG. 8B, delivering the medical device to a treatment site may include exposing the docking station 100 and releasing the docking station 100 from the outer sheath 160. For example, in some variations the outer sheath 160 may be proximally retracted to expose the expandable support 110. Additionally or alternatively, the expandable support 110 may be distally advanced beyond the outer sheath 160 to expose the expandable support 110. As shown in the cross-sectional view of FIG. 8D, in some variations, the expandable support 110 may self-expand as it is exposed, and/or may be expanded with a separate balloon or other device (not shown).

As shown in FIG. 8E, the expandable support 110 may continue to expand until its outer variable diameter apposes the inner wall of the descending aorta. An outer seal may be formed between the expandable support 110 and the descending aorta, thereby substantially prevent fluid (e.g., blood) from passing between the expandable support 110 and the descending aorta. Furthermore, as shown in FIG. 8F, a seal opening 122 may be accessible in the expanded support 110 for receiving a circulatory assist device.

As shown in FIG. 8G, a circulatory assist device 10 (e.g., pVAD) may be positioned axially in the seal opening 120, such that an inflow portion 10a of the circulatory assist device 10 is arranged on a distal side of the support 110 and/or seal 120, and an outflow portion 10b of the circulatory assist device 10 is arranged on a proximal side of the support 110 and/or seal 120. In some instances, as shown in the cross-sectional view of FIG. 8H, the cross-sectional profile of the circulatory assist device 10 may be larger than the initial size of the seal opening 120 (e.g., as shown in FIG. 8F), and the seal opening 120 may dynamically adjust to accommodate the larger size of the circulatory assist device 10. This interference, alone or in combination with the nature of the sealing element(s) (e.g., leaflets) of the seal 120 may facilitate formation of a seal between the circulatory assist device 10 and the seal 120. In some variations, the deployment of the support 110 and/or circulatory assist device 10 at the treatment site in the patient may be performed without imaging guidance (e.g., without X-ray guidance). However, in some variations the deployment of the support 110 and/or circulatory assist device 10 at the treatment site in the patient may be at least partially performed with imaging guidance.

After the circulatory assist device 10 is seated in the seal 120, the circulatory assist device 10 may be operated to receive fluid (e.g., blood) into its inflow region 10a, and pump the received fluid with accelerated momentum toward its outflow region 10b and back into the native aorta lumen, as shown in FIG. 8G. The seal formed between the circulatory assist device 10 and the seal 120 (along with a seal formed between the expandable support 110 and the aortic wall) may substantially prevent backflow of pumped fluid in a proximal-to-distal direction (upwards as shown in FIG. 8G, toward the aortic arch). Accordingly, the use of the docking station 100 in combination with the circulatory assist device 10 may improve the effectiveness of the cardiovascular support provided by the circulatory assist device 10.

In some variations, the method 700 may further include repositioning the circulatory assist device 750. For example, a circulatory assist device may be repositioned among two or more different cardiovascular locations to provide different levels of cardiovascular support (e.g., treatment intensity). For example, the circulatory assist device may be initially placed in the descending aorta (e.g., seated in the docking station) to provide a first level of cardiovascular support, then repositioned to a transvalvular location in which the circulatory assist device is at least partially positioned in the left ventricle to provide second level of cardiovascular support or treatment intensity greater than the first level of cardiovascular support or treatment intensity. For example, the circulatory assist device 10 may be repositioned such that its inflow portion 10a is in the left ventricle, and its outflow portion 10b is in the aorta, such that it extends through the native aortic valve. In this position, the circulatory assist device 10 may be configured to provide a higher level of cardiovascular support compared to when the device 10 is positioned in the descending aorta. When less cardiovascular support is needed by the patient, the circulatory assist device may then be repositioned again in the descending aorta (e.g., seated in the docking station) to tune or adjust the provided cardiovascular support downwards, such as to wean the patient off cardiovascular support. However, in some variations the circulatory assist device may initially be at least partially placed in a left ventricle (and optionally, subsequently repositioned in the descending aorta to tune or adjust the provided cardiovascular support downwards, similar to that described above). Treatment may be escalated and de-escalated repeated by repositioning the circulatory assist device in this manner relative to the docking station 100, as may be clinically desirable for treating the patient.

In some variations, the docking station 100 may remain positioned in the descending aorta for an extended period of time, with or without the circulatory assist device 10 seated therein. For example, in some instances, the docking station 100 with the circulatory assist device 10 may remain in the descending aorta for a first treatment period. As another example, in some instances, the docking station 100 may remain with the circulatory assist device 10 in the descending aorta for a first treatment period, then the docking station 100 may remain in the descending aorta with the circulatory assist device 10 not in the descending aorta (e.g., at least partially in the left ventricle or removed from the patient) for a second treatment period. Following the second treatment period, the docking station 100 may again receive the circulatory assist device 10 therein in the descending aorta for a third treatment period. Alternatively, following the first treatment period or the second treatment period if treatment is no longer desired, the docking station 100 and/or circulatory assist device 10 may be removed from the patient.

In some variations, the docking station 100 may remain in the descending aorta for an extended period of time such as multiple hours, one day, multiple days (e.g., two, three, four, five, or six days), one week, multiple weeks (e.g., two, three, or four weeks), or multiple months (e.g., two, three, or four or more months).

When treatment is no longer desired, the docking station 100 may be removed from the patient. In removing the docking station 100 from the patient, the expandable support 110 and seal 120 may be radially collapsed (e.g., with pull wires, by advancing the outer sheath 160 over the expandable support 110, and/or withdrawing the expandable support 110 back into the outer sheath 160, etc.) and then withdrawn proximally from the patient.

FIGS. 9A-9D illustrate an example method 900 in which the docking station 100 may be decoupled from the elongate member 140 after the docking station 100 is delivered to a treatment site in a patient. FIGS. 9A-9C illustrate delivery of a docking station with an expandable support 110 to a treatment site in a descending aorta. For example, similar to that described above with respect to method 700, a docking station with a support 110 coupled to an elongate member 140 may be advanced to a treatment site in the descending aorta (DA) (FIG. 9A). The support 110 may be exposed by proximally withdrawing the outer sheath 160 and/or distally advancing the support 110 (FIG. 9B), thereby allowing the support 110 to expand (FIG. 9C). As shown in FIGS. 9B and 9C, in some variations the support 110 may be configured to foreshorten (e.g., shorten in length as it radially expands). As shown in FIG. 9D, the elongate member 140 may be decoupled from the support 110, and the elongate member 140 may be removed from the patient, thereby leaving the support 110 and the seal 120 in the descending aorta. Similar to that described above, the docking station 100 may remain in the descending aorta for an extended period of time such as multiple hours, one day, multiple days (e.g., two, three, four, five, or six days), one week, multiple weeks (e.g., two, three, or four weeks), or multiple months (e.g., two, three, or four or more months).

Like the method 700 described herein, the method 900 may further include positioning a circulatory assist device in the seal opening, forming a seal between the circulatory assist device and the seal, and operating the circulatory assist device while the circulatory assist device is positioned in the seal opening. Furthermore, in some variations, the method 900 may further include repositioning the circulatory assist device (e.g., to escalate and/or de-escalate treatment intensity), similar to that described above with respect to method 700.

When treatment is no longer desired, the docking station 100 may be removed from the patient. In removing the docking station 100 from the patient, the expandable support 110 and seal 120 may be radially collapsed and/or withdrawn from the patient, such as by engaging with an engagement feature 130 on the support 110 with a retrieval tool. The expandable support 110 may then be withdrawn proximally and removed from the patient.

### Conclusion

Although many of the variations are described above with respect to systems, devices, and methods for providing circulatory assistance, the technology is applicable to other applications and/or other approaches. Moreover, other variations in addition to those described herein are within the scope of the technology. Additionally, several other variations of the technology can have different configurations, components, or procedures than those described herein. A person of ordinary skill in the art, therefore, will accordingly understand that the technology can have other variations with additional elements, or the technology can have other variations without several of the features shown and described above with reference to FIGS. 1A-13B.

The descriptions of variations of the technology are not intended to be exhaustive or to limit the technology to the precise form disclosed above. Where the context permits, singular or plural terms may also include the plural or singular term, respectively. Although specific variations of, and examples for, the technology are described above for illustrative purposes, various equivalent modifications are possible within the scope of the technology, as those skilled in the relevant art will recognize. For example, while steps are presented in a given order, alternative variations may perform steps in a different order. The various variations described herein may also be combined to provide further variations.

As used herein, the terms “generally,” “substantially,” “about,” and similar terms are used as terms of approximation and not as terms of degree, and are intended to account for the inherent variations in measured or calculated values that would be recognized by those of ordinary skill in the art.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific variations have been described herein for purposes of illustration, but that various modifications may be made without deviating from the technology. Further, while advantages associated with certain variations of the technology have been described in the context of those variations, other variations may also exhibit such advantages, and not all variations need necessarily exhibit such advantages to fall within the scope of the technology. Accordingly, the disclosure and associated technology can encompass other variations not expressly shown or described herein.

1. A medical device, comprising:
an expandable support having a delivery configuration with a first diameter and a deployed configuration with a second diameter larger than the first diameter, wherein the expandable support comprises an interior region; and
a seal arranged within the interior region of the expandable support, wherein the seal comprises a seal opening and a sealing element configured to seal around a circulatory assist device seated in the seal opening.

2. The medical device of claim 1, wherein the seal opening has a variable diameter.

3. The medical device of claim 1, wherein the seal opening has a fixed diameter.

4. The medical device of claim 1, wherein the seal opening is radially centered within the interior region of the expandable support.

5. The medical device of claim 1, wherein the seal opening is radially offset within the interior region of the expandable support.

6. The medical device of claim 1, wherein the sealing element comprises a plurality of leaflets.

7. The medical device of claim 1, wherein the seal comprises a valve.

8. (canceled)

9. The medical device of claim 1, wherein the expandable support is self-expandable.

10. The medical device of claim 9, wherein the expandable support comprises a stent.

11-12. (canceled)

13. The medical device of claim 1, wherein the expandable support comprises a shape memory material.

14. The medical device of claim 1, wherein the expandable support comprises an inflatable member.

15. The medical device of claim 1, further comprising a first pressure sensor configured to measure a pressure on an upstream side of the seal, and a second pressure sensor configured to measure a pressure on a downstream side of the seal.

16. The medical device of claim 1, further comprising an outer sheath comprising a lumen configured to hold the expandable support in the delivery configuration.

17. The medical device of claim 1, further comprising an elongate member coupled to the expandable support configured for one or more of delivery or retrieval of the medical device.

18. The medical device of claim 17, wherein the elongate member is removably coupled to the expandable support.

19. The medical device of claim 1, wherein the expandable support comprises an engagement feature configured to facilitate retrieval of the medical device from a treatment site.

20. The medical device of claim 19, wherein the engagement feature comprises at least one of a knob, loop, hook, or mating feature, coupled to a downstream surface of the expandable support.

21. The medical device of claim 1, wherein the expandable support comprises a bioabsorbable material.

22. The medical device of claim 1, wherein the expandable support is configured to transition to a flow restoration configuration in which a diameter of the interior region is larger than the seal opening.

23-27. (canceled)

28. A method of providing circulatory assistance, comprising:
delivering a medical device to a treatment site in a vascular lumen of a patient, wherein the medical device comprises an expandable support and a seal arranged within an interior region of the expandable support, wherein the seal comprises a seal opening;
positioning a circulatory assist device in the seal opening;
forming a seal between the circulatory assist device and the expandable support with the seal; and
operating the circulatory assist device while the circulatory assist device is positioned in the seal opening.

29-60. (canceled)
